(19) United States
(12) Patent Application Publication
(10) Pub. No.: US 2002/0061861 A1
(43) Pub. Date: May 23, 2002

(54) Nucleic acid expression from linear nucleic acids

(76) Inventors: Hans Herweijer, Madison, WI (US); Christine I. Wooddell, Madison, WI (US)

Correspondence Address: Mark K. Johnson, PO Box 510644, New Berlin, WI, 53151-0644, US

(21) Appl. No.: 09/932,521

(22) Filed: Aug. 17, 2001

Related U.S. Application Data
(60) Provisional application No. 60/225,946, filed on Aug. 17, 2000.

(51) Int. Cl. A61K 48/00; C12P 19/34; C12N 15/87

(52) U.S. Cl. 514/44; 435/91.2; 435/455; 435/320.1

(57) ABSTRACT

The present invention relates to processes for delivering an expression cassette, comprised of a linear nucleic acid capable of expressing a gene or partial gene, and expressing the gene or partial gene. The linear nucleic acid is delivered to the host, where the nucleic acid transfects host cells resulting in expression of the encoded gene product. This invention allows the encoded gene product to be expressed for long periods of time.

This application is related to and claims priority benefit of U.S. Provisional Application Serial No. 60/225,946 filed on Aug. 17, 2000.

### FIELD

This invention relates to compositions and methods for nucleic acid delivery and expression. More particularly, this invention relates to nucleic acids enabling long term gene expression. In preferred embodiments, methods for generation of nucleic acids enabling long term gene expression are disclosed.

### BACKGROUND

Delivery and Vector Development

DNA transfer, for both direct and indirect approaches, can be accomplished by viral and non-viral delivery. Non-viral methods include polylysine conjugates, various polymers such as PEI, liposomes, cationic lipids, the biolistic “gun”, and naked DNA. Viral methods include adenovirus, adeno-associated virus (AAV), retrovirus and lentivirus vectors. Despite the great promise of gene delivery, the formidable challenge of efficiently transferring and stably expressing transgenes in cells remains. Another problem area in gene delivery is the sustained expression of transgenes at high levels in tissues such as the liver, pulmonary epithelium and muscle.

Advantages of Non-Viral Vectors

The relative merits of the two types of direct gene delivery, viral and non-viral, is under active investigation as rapid advances are being made in each of these two technologies. Nonetheless, plasmid-based vectors appear to offer some advantages over viral vectors aside from efficiency. Some viral vectors, such as herpes or adenoviral vectors, may retain viral promoters and genes that could express in human cells under certain conditions, causing immune or other adverse effects. On the other hand, studies indicate that non-human primates do not produce anti-DNA antibodies, even after repetitive administrations of naked plasmid DNA [1]. Viral vectors are also difficult to scale up for human use. In contrast, plasmid DNA can be scaled-up easily in large culture vessels. Improvements in plasmid purification by column chromatography are further reducing the cost of plasmid preparation. The recent death of a patient following adenoviral gene delivery has generated greater interest in non-viral vectors.

The decreased efficiency of gene transfer relative to viral vectors is becoming less of a problem. For example, the intravascular delivery of naked pDNA into muscle [2, 3] and liver [4, 5] tissues can lead to expression in up to 20% of the cells, approaching the levels achieved by viruses. A variety of other non-viral vectors systems are also likely to lead to great strides in the efficiency of non-viral gene transfer. These include liposome (lipoplexes), polymer (polyplexes), and combination of these (lipopolyplexes). The great strides in raising the efficiency of transfection in cultured cells in vitro over the past several years supports this contention. For example, TransIT products (produced by Mirus and distributed through PanVera, Fisher, and Takara) can transfect 100% of some cell lines, whereas the first cationic lipid (Lipofectin, Life Technologies) has substantially less transfection efficiency.

Significance of Non-Viral Integration Systems

One objective of gene delivery research is the development of a non-viral procedure to effect stable expression at useful levels. The ability of retroviral and adeno-associated virus vectors to integrate into mammalian genomes increases their utility for enabling prolonged expression in dividing cells. However, these vectors have limited insert capacity: Retrovirus and AAV vectors can only carry up to ten and five kilobases of foreign DNA, respectively. This not only seriously limits the size of cDNA's that can be expressed (e.g., dystrophin for Duchenne muscular dystrophy) but also restricts the size of the regulatory sequences. The ability to use large genes with almost complete transcriptional and translational cis regulatory sequences would aid the development of non-viral gene cassettes for high and stable expression. The full complement of regulatory sequences would also enable the expression of foreign genes to be under better physiologic, tissue-specific, and developmental control. For example, a 12-kb fragment of the 5′-flanking region of the albumin gene was shown to enable higher levels of liver expression than the 0.3-kb fragment that is commonly used [6]. Furthermore, viral cis sequences (e.g., retroviral LTR sequences) can also adversely affect foreign gene expression. In addition, the production of viral vectors is laborious and limits the number of constructs and regulatory sequences that can be evaluated.

While integration may be desired, non-specific integration can also be considered a drawback in terms of inactivating genes required for cell viability or activating proto-oncogenes. The cellular toxicity resulting from gene inactivation is considered inconsequential given the rarity of the event and the insignificance of losing a few cells. On the other hand, the potential danger of causing tumors has been considered more seriously in terms of retroviral vectors. The experimental evidence is that tumor promotion has only been observed when high titers of replication competent virus were maintained in the blood of non-human primates. The requirement for several cellular steps for neoplastic transformation is probably why a few integration events per cell do not lead to cancer. The late Howard Temin has argued strongly for this position. In addition, the proto-oncogenes would be turned on by the presence of promoters in the 3′ LTR. This would not be the case for pDNA vectors. Additional safety could be engineered into the pDNA vectors by the insertion of transcription termination signals to prevent read-through.

For direct approaches, the ability to repetitively administer a vector, such as naked pDNA, enables incremental increases in gene expression. If one administration does not provide sufficient expression for efficacy then multiple administrations would provide the required amount of gene product. In addition, repetitive administration of an integrating vector enables gene transfer and expression to be titrated so as to produce the gene product (e.g., erythropoietin) within a window.

Liver DNA delivery

We are particularly interested in applying non-viral gene transfer methods into hepatocytes given the central role that the liver plays in many inborn errors of metabolism and acquired disorders such as hemophilia, hypercholesterolemia, and hepatitis. A variety of techniques have been developed to transfer genes into the liver. Cultured hepatocytes have been genetically modified by retroviral vectors and implanted back into livers of animals and humans. Retroviral vectors have also been delivered directly to livers in which hepatocyte division was induced by partial hepatectomy. Injection of adenoviral vectors into the portal or tail vein leads to high levels of foreign gene expression in the liver that is transient. More long term expression has been achieved using a gutted adenoviral vector. Recently, it has been reported that long-term expression in liver occurred from adeno-associated virus (AAV) vectors and lentiviral vectors. Non-viral transfer methods have included polycation complexes of asialoglycoproteins that are systemically administered.

The injection of naked plasmid DNA (pDNA) into liver or tail vein vessels leads to high levels of foreign gene expression in rodent hepatocytes. Almost milligram quantities of foreign protein could be produced in over 5% of the hepatocytes one day after injection[7]. Such levels of gene transfer are sufficient to treat several common genetic diseases. For instance, in hemophilia A and B (Factor VIII and IX deficiency, respectively) the clinical course of the disease is greatly influenced by the percentage of the normal serum levels of factor VIII or IX: <2%, severe; 2-5%, moderate; and 5-30%, mild. This indicates that in severe patients gene therapy yielding only 2% of the normal level can be of great help. Levels greater than 6% prevent spontaneous bleeding, but not those secondary to injury or surgery. Preliminary experiments have shown that intravascular injection of pCI-hFIX (an expression vector in which the human factor IX gene is under transcriptional control of the cytomegalovirus promoter) in mice resulted in serum levels of ˜3,500 ng/ml after one day. This is equivalent to ˜70% of the normal human serum levels and suggests that the efficiency of the current naked DNA in vivo transfection method would be sufficient for human gene therapy for several genetic diseases (hemophilia, phenylketonuria, hypercholesterolemia, urea cycle disorders and organic acidurias) if expression levels were stable.

Unfortunately, the peak levels of expression from naked DNA and other non-viral gene transfer methods can not be maintained, thereby limiting their clinical utility. The reasons for this loss of expression are complicated and under investigation. An early phase within the first few days appear to be due to transcriptional down-regulation. A later phase of decreasing gene expression is probably due to an immune response directed against the expressed transgene.

Delivery of Nucleic Acids

We have described a very efficient method for plasmid DNA gene transfer into murine liver[7, 8]. High levels of expression in hepatocytes could be achieved after intraportal delivery of plasmid DNA vectors with up to 10% of all liver cells transfected. Gene transfer efficiency into hepatocytes is increased by injections under pressure and by raising the osmolarity of the injection solution. This is achieved by the use of 15% (w/v) mannitol in the injection solution. The use of fluorescently-labeled pDNA indicates that these conditions enable the extravasation of the pDNA, perhaps through disruption of tight junctions or an increase in sinusoid fenestrae size. High volume, high pressure tail vein injections [5] allow for very efficient delivery of pDNA to the liver (and with lower efficiency to other organs).

This simple, highly efficient procedure allows for the rapid and efficient testing of novel elements in vivo, avoiding the laborious and costly production of transgenic animals. It should be noted that intravascular delivery of pDNA to the liver of larger animals (e.g., rat, dog) is also possible [8].

An intravascular route of administration enables a polymer or polynucleotide to be delivered to cells more evenly distributed and more efficiently expressed than direct injections. Intravascular herein means within a tubular structure called a vessel that is connected to a tissue or organ within the body. Within the cavity of the tubular structure, a bodily fluid flows to or from the body part. Examples of bodily fluid include blood, lymphatic fluid, or bile. Examples of vessels include arteries, arterioles, capillaries, venules, sinusoids, veins, lymphatics, and bile ducts. The intravascular route includes delivery through the blood vessels such as an artery or a vein. Patent number (U.S. patent application Ser. No. 08/975,573) incorporated herein by reference. An administration route involving the mucosal membranes is meant to include nasal, bronchial, inhalation into the lungs, or via the eyes.

In Vivo Transfection Reagents

Previously-developed non-viral particles aggregate in physiologic solutions. The large size of these aggregates interferes with their ability to transfect cells in vivo. In addition, previously-developed non-viral particles required a net positive charge in order for the packaged DNA to be fully protected. However, particles with a net positive charge interact non-specifically with many blood and tissue components, thereby preventing their contact with target cells in vivo. Furthermore, currently-available preparations contain a harmful excess of free polymer, which can be removed from our particles. In summary, the inability to encase DNA into virus-like, artificial particles that are neutral or negatively-charged, and that do not aggregate have greatly hampered the efficiency and thus the utility of non-viral gene delivery. This problem in constructing DNA particles has been solved by us.

We have developed a new method for constructing DNA supramolecular complexes. It entails the formation of polymers on DNA, a process termed template polymerization [9]. It greatly expands the range of tools that can be used for the construction of gene transfer particles. Conceptually, it is a “nanotechnology” and a “synthetic self-assembling system.” The process mimics biologic processes of supramolecular assembly, which often involves template polymerization. The gene complexes formed by DNA template polymerization are ideal for direct, non-viral gene delivery because they do not aggregate in physiologic solution, and are small (<70 nm).

We have shown condensation of pDNA into small particles that are stable under physiological conditions [10]. It allows for “recharging,” i.e., the formation of negatively charged particles that do not bind non-specifically to cells in vivo. Upon cell internalization, these particles release the pDNA, allowing nuclear uptake and expression. Ligands, endosomal release-enhancing groups, nuclear localizing signals, and other moieties can be attached to these particles through simple chemistry. Altogether, this platform technology allows for highly-efficient, cell type-specific transfections in vivo.

In template polymerization (TP), cationic monomers, having an inherent electrostatic attraction for DNA, are polymerized (cross-linked) along a DNA template. Chain and step polymerization processes can be used to form DNA complexes using distinct types of cationic monomers for each process. Chain polymerization involves the successive addition of monomer units to a limited number of growing polymer chains. The polymerization rate remains constant until the monomer concentration is depleted. Monomers containing vinyl, acrylate, methacrylate, acrylamide, and methacrylamide groups undergo chain polymerization. Polymerization is initiated by radical, anionic, or cationic processes. Some of these monomers are pH-sensitive and bear a positive charge only within a certain pH range.

Another of our technologies, “DNA caging,” is a specific type of TP that prevents aggregation of DNA particles by starting with macromonomers (i.e., polycations of molecular weight>10,000)[10]. This technology comprises the treatment of preformed DNA/polycation complexes with a cleavable bifunctional reagent so DNA becomes entrapped (caged) inside a cross-linked net of counter-ions. If cross-linkers bearing positive charge were used (such as bis-imido esters) the resulting complexes stay soluble even at high salt concentrations in conditions where non-caged complexes flocculate. Caged particles are stable in physiological salt but also contain labile groups that enable the particles to disassemble in cells.

Another component comprises the preparation of negatively charged (“recharged”) particles of condensed DNA by coating them with polyanions[10]. In addition, the polyanions can be designed to carry cell-specific moieties to enhance tissue targeting. Because the pDNA is caged within a polycation layer, the outside layer of polyanions cannot displace the pDNA. This procedure represents a unique opportunity to design small and negatively charged particles of condensed pDNA. In addition, excess polymer can be removed from the caged and recharged particles using size exclusion chromatography. Preliminary results indicate that these recharged particles can transfect hepatocytes in vivo as efficiently as naked DNA.

A major part of our research is focussed on the synthesis of novel polyions and polyions with ligands, forming stable pDNA particles, and evaluating these particles in vitro and in vivo for stability, targeting specificity, and transfection efficiency. Our current efforts have mainly dealt with intravascular delivery to target liver and muscle cells. We have successfully attached several ligands to polycations and polyanions (e.g., galactose, folate, transferrin).

Linear DNA

It is customary to use covalently closed circular (usually supercoiled) plasmid DNA for gene transfer and expression. Yet, it is possible to use linearized pDNA as well. pDNA can be linearized by using restriction enzymes. Such restriction enzymes can leave short overhangs (sticky ends), or leave no overhangs (blunt ends). A pDNA can be digested at one single site, thus leaving all pDNA elements in place in the linear DNA. Restriction at multiple sites (with one or more enzymes) allows the generation of expression cassettes devoid of some of the pDNA elements. For example, the bacterial drug resistance gene may be deleted while leaving the expression cassette intact. Such expression cassettes can also be generated by polymerase chain reaction (PCR).

### References

1. S. Jiao, G. Aesadi, A. Jani, P. L. Felgner and J. A. Wolff. Exp Neurol 115:400-413, 1992.

2. V. Budker, G. Zhang, I. Danko, P. Williams and J. Wolff. Gene Ther. 5:272-276, 1998.

3. G. Zhang, V. Budker, P. Williams, V. Subbotin and J. A. Wolff. Hum Gene Ther 12:427-438., 2001.

4. F. Liu, Y. K. Song and D. Liu. Gene Therapy 6:1258-1266, 1999.

5. G. Zhang, V. Budker and J. A. Wolff. Human Gene Therapy 10:1735-1737, 1999.

6. C. A. Pinkert, D. M. Ornitz, R. L. Brinster and R. D. Palmiter. Genes Dev 1:268-276, 1987.

7. V. Budker, G. Zhang, S. Knechtle and J. A. Wolff. Gene Ther. 3:593-598, 1996.

8. G. Zhang, D. Vargo, V. Budker, N. Armstrong, S. Knechtle and J. A. Wolff. Hum. Gene Ther. 8:1763-1772, 1997.

9. V. S. Trubetskoy, V. G. Budker, L. J. Hanson, P. M. Slattum, J. A. Wolff and J. E. Hagstrom. Nucleic Acids Res 26:4178-4185, 1998.

10. V. S. Trubetskoy, A. Loomis, P. M. Slattum, J. E. Hagstrom, V. G. Budker and J. A. Wolff. Bioconjug Chem 10:624-628, 1999.

11. Z. Y. Chen, S. R. Yant, C. Y. He, L. Meuse, S. Shen and M. A. Kay. Mot Ther 3:403-410., 2001.

### SUMMARY OF THE INVENTION

The present invention relates to compositions and methods for expressing nucleic acids in cells in vivo following non-viral gene transfer. The nucleic acid is linear.

In one aspect, the present invention provides a composition consisting of a nucleic acid encoding a gene under control of regulatory sequences appropriate for the target cell and host.

In some preferred embodiments the nucleic acid expresses a gene.

In some preferred embodiments the nucleic acid expresses a partial gene.

In some preferred embodiments the linear nucleic acid has blunt ends.

In some preferred embodiments the linear nucleic acid has sticky ends.

In some preferred embodiments the linear nucleic acid has one blunt end and one sticky end.

In some preferred embodiments the nucleic acid is linearized by restriction enzyme digestion.

In some preferred embodiments the linear nucleic acid is synthesized by the polymerase chain reaction process.

In some preferred embodiments the linear expression cassette is isolated from plasmid backbone sequences.

In some preferred embodiments the expression cassette is flanked by ends derived from transposases.

In some preferred embodiments the expression cassette is flanked by ends derived from Tn5 transposase.

In some preferred embodiments the expression cassette is flanked by the outside ends derived from Tn5 transposase.

In some preferred embodiments the expression cassette is flanked by the inside ends derived from Tn5 transposase.

In some preferred embodiments the expression cassette is flanked by the chimeric ends derived from Tn5 transposase.

In another aspect, the nucleic acid is complexed with a polymer.

In some preferred embodiments, the delivery system comprises injecting the nucleic acid or nucleic acid-polymer complexes intravascularly.

In some preferred embodiments, the delivery system comprises injecting the nucleic acid or nucleic acid-polymer complexes intravascularly under elevated pressure.

In some preferred embodiments, the delivery system comprises direct intramuscular injection of the nucleic acid or nucleic acid-polymer complexes.

In some preferred embodiments, the delivery system comprises nucleic acid or nucleic acid-polymer complexes delivered to the intestines.

In some preferred embodiments, the delivery system comprises direct interstitial injection of the nucleic acid or nucleic acid-polymer complexes.

### BRIEF DESCRIPTION OF THE DRAWINGS

FIG. 1 is a graph illustrating Human factor IX expression from linearized DNA templates. Mice were injected into the tail vein with 10 μg either supercoiled or blunt-end linearized plasmid pMIR7 encoding human factor IX. Human factor IX (ng/ml) was measured in the plasma from mice at the indicated days post injection. Expression levels are shown for two of the mice injected with the linearized pMIR7. Expression levels at day one from mice injected with supercoiled or linearized pMIR7 were comparable, but there was no detectable expression after day 7 from the mice injected with supercoiled pMIR7.

### DETAILED DESCRIPTION OF THE INVENTION

I. Definitions

To facilitate an understanding of the present invention, a number of terms and phrases are defined below:

The term “nucleic acid” is a term of art that refers to a polymer containing at least two nucleotides. “Nucleotides” contain a sugar deoxyribose (DNA) or ribose (RNA), a base, and a phosphate group. Nucleotides are the monomeric units of nucleic acid polymers. Nucleotides are linked together through the phosphate groups to form nucleic acid. A “polynucleotide” is distinguished here from an “oligonucleotide” by containing more than 100 monomeric units; oligonucleotides contain from 2 to 100 nucleotides. “Bases” include purines and pyrimidines, which further include natural compounds adenine, thymine, guanine, cytosine, uracil, inosine, and other natural analogs, and synthetic derivatives of purines and pyrimidines, which include, but are not limited to, modifications which place new reactive groups such as, but not limited to, amines, alcohols, thiols, carboxylates, and alkylhalides. The term nucleic acid includes deoxyribonucleic acid (“DNA”) and ribonucleic acid (“RNA”). The term nucleic acid encompasses sequences that include any of the known base analogs of DNA and RNA including, but not limited to, 4-acetylcytosine, 8-hydroxy-N6-methyladenosine, aziridinylcytosine, pseudoisocytosine, 5-(carboxyhydroxylmethyl) uracil, 5-fluorouracil, 5 -bromouracil, 5-carboxymethylaminomethyl-2-thiouracil, 5-carboxymethylaminomethyluracil, dihydrouracil, inosine, N6-isopentenyladenine, 1-methyladenine, 1-methylpseudouracil, 1-methylguanine, 1-methylinosine, 2,2-dimethyl-guanine, 2-methyladenine, 2-methylguanine, 3-methylcytosine, 5-methylcytosine, N6-methyladenine, 7-methylguanine, 5-methylaminomethyluracil, 5-methoxyamino-methyl-2-thiouracil, beta-D-mannosylqueosine, 5′-methoxycarbonylmethyluracil, 5-methoxyuracil, 2-methylthio-N6-isopentenyladenine, uracil-5-oxyacetic acid methylester, uracil-5-oxyacetic acid, oxybutoxosine, pseudouracil, queosine, 2-thiocytosine, 5-methyl-2-thiouracil, 2-thiouracil, 4-thiouracil, 5-methyluracil, N-uracil-5-oxyacetic acid methylester, uracil-5-oxyacetic acid, and 2,6-diaminopurine.

Nucleic acids may be linear, circular, or have higher orders of topology (e.g., supercoiled plasmid DNA). DNA may be in the form of anti-sense, plasmid DNA, parts of a plasmid DNA, vectors (PI, PAC, BAC, YAC, artificial chromosomes), expression cassettes, chimeric sequences, chromosomal DNA, or derivatives of these groups. RNA may be in the form of oligonucleotide RNA, tRNA (transfer RNA), snRNA (small nuclear RNA), rRNA (ribosomal RNA), mRNA (messenger RNA), anti-sense RNA, (interfering) double stranded RNA, ribozymes, chimeric sequences, or derivatives of these groups. “Anti-sense” is a nucleic acid that interferes with the function of DNA and/or RNA. This may result in suppression of expression. Interfering RNA (“RNAi”) is double stranded RNA that results in catalytic degradation of specific mRNAs, and can also be used to lower gene expression. Natural nucleic acids have a phosphate backbone; artificial nucleic acids may contain other types of backbones, nucleotides, or bases. Artificial nucleic acids with modified backbones include peptide nucleic acids (PNAs), phosphothionates, phosphorothioates, phosphorodiamidate morpholino, and other variants of the phosphate backbone of native nucleic acids.

Examples of modified nucleotides include methylation, mustard addition, and aromatic nitrogen mustard addition. “Mustards” include nitrogen mustards and sulfur mustards. Mustards are molecules consisting of a nucleophile and a leaving group separated by an ethylene bridge. After internal attack of the nucleophile on the carbon bearing the leaving group a strained three membered group is formed. This strained ring (in the case of nitrogen mustards an aziridine ring is formed) is very susceptible to nucleophilic attack, thus allowing mustards to alkylate weak nucleophiles such as nucleic acids. Mustards can have one of the ethylene bridged leaving groups attached to the nucleophile, these molecules are sometimes referred to as half-mustards; or they can have two of the ethylene bridged leaving groups attached to the nucleophile, these molecules can be referred to as bis-mustards. A “nitrogen mustard” is a molecule that contains a nitrogen atom and a leaving group separated by an ethylene bridge, i.e. R₂NCH₂CH₂X where R=any chemical group, and X=a leaving group typically a halogen. An “aromatic nitrogen mustard” is represented by RR′NCH₂CH₂X (wherein R=any chemical group, N=nitrogen, X=a leaving group, typically a halogen, R′=an aromatic ring, R=any chemical group).

Nucleic acid may be single (“ssDNA”), double (“dsDNA”), triple (“tsDNA”), or quadruple (“qsDNA”) stranded DNA, and single stranded RNA (“RNA”) or double stranded RNA (“dsRNA”). “Multistranded” nucleic acid contains two or more strands and can be either homogeneous as in double stranded DNA, or heterogeneous, as in DNA/RNA hybrids. Multistranded nucleic acid can be full length multistranded, or partially multistranded. It may further contain several regions with different numbers of nucleic acid strands. Partially single stranded DNA is considered a sub-group of ssDNA and contains one or more single stranded regions as well as one or more multiple stranded regions.

“Enzymatic reaction” refers to processes mediated by enzymes. Enzymatic reactions can also be used to generate single-stranded DNA. One strand of a double stranded nucleic acid can be preferentially degraded into nucleotides using a nuclease. Many ribonucleases are known with specific activity profiles that can be used for such a process. For instance, RNase H can be used to specifically degrade the RNA strand of an RNA-DNA double stranded hybrid nucleic acid, which in itself may have been formed by the enzymatic reaction of reverse transcriptase synthesizing the DNA stranded using the RNA strand as the template. Following the introduction of a nick, a ribonuclease can specifically degrade the strand with the nick, generating a partially single stranded nucleic acid. A RNA or DNA dependent DNA polymerase can synthesize new DNA which can subsequently be isolated (e.g., by denaturation followed by separation). The polymerase chain reaction process can be used to generate nucleic acids. Formation of single stranded nucleic acid can be favored by adding one oligonucleotide primer in excess over the other primer (“asymmetric PCR”). Alternatively, one of the DNA strands formed in the PCR process may be separated from the other (e.g., by using a ligand in one of the primers).

“Restriction enzymes” are enzymes of bacterial or viral origin that cut DNA at palindromic sequences. Each restriction enzyme has a specific recognition sequence. These sequences are usually 4 to 8 base-pairs. There are hundreds of restriction sites in a typical plasmid; some of these sites are frequent and others infrequent. Restriction enzymes can be used to generate DNA with blunt ends or ends that have one of the strands overhanging the other (“sticky” ends).

“Expression cassette” refers to a natural or recombinantly produced nucleic acid molecule that is capable of expressing protein(s). A DNA expression cassette typically includes a promoter (allowing transcription initiation), and a sequence encoding one or more proteins. Optionally, the expression cassette may include trancriptional enhancers, non-coding sequences, splicing signals, transcription termination signals, and polyadenylation signals. An RNA expression cassette typically includes a translation initiation codon (allowing translation initiation), and a sequence encoding one or more proteins. Optionally, the expression cassette may include translation termination signals, a polyadenosine sequence, internal ribosome entry sites (IRES), and non-coding sequences. A nucleic acid can be used to modify the genomic or extrachromosomal DNA sequences. This can be achieved by delivering a nucleic acid that is expressed. Alternatively, the nucleic acid can effect a change in the DNA or RNA sequence of the target cell. This can be achieved by hybridization, multistrand nucleic acid formation, homologous recombination, gene conversion, or other yet to be described mechanisms.

The term “gene” generally refers to a nucleic acid sequence that comprises coding sequences necessary for the production of a therapeutic nucleic acid (e.g., ribozyme) or a polypeptide or precursor. The polypeptide can be encoded by a full length coding sequence or by any portion of the coding sequence so long as the desired activity or functional properties (e.g., enzymatic activity, ligand binding, signal transduction) of the full-length polypeptide or fragment are retained. The term also encompasses the coding region of a gene and the including sequences located adjacent to the coding region on both the 5′ and 3′ ends for a distance of about 1 kb or more on either end such that the gene corresponds to the length of the full-length mRNA. The sequences that are located 5′ of the coding region and which are present on the mRNA are referred to as “5′ untranslated sequences.” The sequences that are located 3′ or downstream of the coding region and which are present on the mRNA are referred to as “3′ untranslated sequences.” The term gene encompasses both cDNA and genomic forms of a gene. A genomic form or clone of a gene contains the coding region interrupted with “non-coding sequences” termed “introns” or “intervening regions” or “intervening sequences.” Introns are segments of a gene which are transcribed into nuclear RNA. Introns may contain regulatory elements such as enhancers. Introns are removed or “spliced out” from the nuclear or primary transcript; introns therefore are absent in the messenger RNA (mRNA) transcript. The mRNA functions during translation to specify the sequence or order of amino acids in a nascent polypeptide. The term non-coding sequences also refers to other regions of a genomic form of a gene including, but not limited to, promoters, enhancers, transcription factor binding sites, polyadenylation signals, internal ribosome entry sites, silencers, insulating sequences, matrix attachment regions. These sequences may be present close to the coding region of the gene (within 10,000 nucleotide) or at distant sites (more than 10,000 nucleotides). These non-coding sequences influence the level or rate of transcription and translation of the gene. Covalent modification of a gene may influence the rate of transcription (e.g., methylation of genomic DNA), the stability of mRNA (e.g., length of the 3′ polyadenosine tail), rate of translation (e.g., 5′ cap), nucleic acid repair, and immunogenicity. One example of covalent modification of nucleic acid involves the action of LabelIT reagents (Mirus Corporation, Madison, Wis.).

As used herein, the terms “nucleic acid molecule encoding,” “DNA sequence encoding,” and “DNA encoding” refer to the order or sequence of deoxyribonucleotides along a strand of deoxyribonucleic acid. The order of these deoxyribonucleotides determines the order of amino acids along the polypeptide (protein) chain. The DNA sequence thus codes for the amino acid sequence. As used herein, the terms “an oligonucleotide having a nucleotide sequence encoding a gene,” “a polynucleotide having a nucleotide sequence encoding a gene,” and “a nucleic acid having a nucleotide sequence encoding a gene,” mean a nucleic acid sequence comprising the coding region of a gene or in other words the nucleic acid sequence which encodes a gene product. The coding region may be present in either a cDNA, genomic DNA or RNA form. When present in a DNA form, the nucleic acid may be single-stranded, double-stranded, multistranded, partially single stranded, or partially multistranded. Suitable control elements such as, but not limited to, enhancers/promoters, splice junctions, and polyadenylation signals, may be placed in close proximity to the coding region of the gene if needed to permit proper initiation of transcription and correct processing of the primary RNA transcript. Alternatively, the coding region utilized in the expression vectors may contain endogenous enhancers/promoters, splice junctions, intervening sequences, polyadenylation signals; exogenous control elements; or a combination of both endogenous and exogenous control elements.

The term “isolated” when used in relation to a nucleic acid, as in “an isolated nucleic acid” refers to a nucleic acid sequence that is identified and separated from at least one contaminant nucleic acid with which it is ordinarily associated in its natural source. Isolated nucleic acid is present in a form or setting that is different from that in which it is found in nature. In contrast, “non-isolated nucleic acids” are nucleic acids, such as DNA and RNA, found in the state they exist in nature. For example, a given DNA sequence (e.g., a gene) is found on the host cell chromosome in proximity to neighboring genes; RNA sequences, such as a specific mRNA sequence encoding a specific protein, are found in the cell as a mixture with numerous other mRNAs that encode a multitude of proteins. However, isolated nucleic acid encoding a given protein includes, by way of example, such nucleic acid in cells ordinarily expressing the given protein where the nucleic acid is in a chromosomal location different from that of natural cells, or is otherwise flanked by a different nucleic acid sequence than that found in nature. The isolated nucleic acid may be present in single stranded, partially single stranded, multistranded, or partially multistranded form.

As used herein, the term “gene expression” refers to the process of converting genetic information encoded in a gene into RNA (e.g., mRNA, rRNA, tRNA, or snRNA) through “transcription” of a deoxyribonucleic gene (e.g., via the enzymatic action of an RNA polymerase), and for protein encoding genes, into protein through “translation” of mRNA. Gene expression can be regulated at many stages in the process. “Up-regulation” or “activation” refers to regulation that increases the production of gene expression products (i.e., RNA or protein), while “down-regulation” or “repression” refers to regulation that decreases production. Molecules (e.g., transcription factors) that are involved in up-regulation or down-regulation are often called “activators” and “repressors,” respectively.

Expression of a gene from a linear nucleic acid for an “extended period of time” is defined as expression for longer than 7 days with at least 20% more gene product than is expressed from the supercoiled plasmid that the linear nucleic acid derives from.

Two molecules are combined, to form a “complex” through a process called “complexation” or “complex formation,” if the are in contact with one another through “non-covalent” interactions such as, but not limited to, electrostatic interactions, hydrogen bonding interactions, and hydrophobic interactions. An “interpolyelectrolyte complex” is a non-covalent interaction between polyelectrolytes of opposite charge. A molecule is “modified,” through a process called “modification,” by a second molecule if the two become bonded through a covalent bond. That is, the two molecules form a covalent bond between an atom from one molecule and an atom from the second molecule resulting in the formation of a new single molecule. A chemical “covalent bond” is an interaction, bond, between two atoms in which there is a sharing of electron density.

The terms “naked nucleic acid” and “naked polynucleotide” indicate that the nucleic acid or polynucleotide is not associated with a transfection reagent or other delivery vehicle that is required for the nucleic acid or polynucleotide to be delivered to the cell. A “transfection reagent” is a compound or compounds that bind(s) to or complex(es) with oligonucleotides and polynucleotides, and mediates their entry into cells. The transfection reagent also mediates the binding and internalization of oligonucleotides and polynucleotides into cells. Examples of transfection reagents include cationic liposomes and lipids, polyamines, calcium phosphate precipitates, histone proteins, polyethylenimine, and polylysine complexes. It has been shown that cationic proteins like histones and protamines, or synthetic polymers like polylysine, polyarginine, polyornithine, DEAE dextran, polybrene, and polyethylenimine may be effective intracellular delivery agents, while small polycations like spermine are ineffective. Typically, the transfection reagent has a net positive charge that binds to the oligonucleotide's or polynucleotide's negative charge. The transfection reagent mediates binding of oligonucleotides and polynucleotides to cells via its positive charge (that binds to the cell membrane's negative charge) or via ligands that bind to receptors in the cell. For example, cationic liposomes or polylysine complexes have net positive charges that enable them to bind to DNA or RNA. Polyethylenimine, which facilitates gene transfer without additional treatments, probably disrupts endosomal function itself.

Other vehicles are also used, in the prior art, to transfer genes into cells. These include complexing the nucleic acids on particles that are then accelerated into the cell. This is termed “biolistic” or “gun” techniques. Other methods include electroporation, microinjection, liposome fusion, protoplast fusion, viral infection, and iontophoresis.

“Intravascular” refers to an intravascular route of administration that enables a polymer, oligonucleotide, or polynucleotide to be delivered to cells more evenly distributed and more efficiently than direct injections. Intravascular herein means within an internal tubular structure called a vessel that is connected to a tissue or organ within the body of an animal, including mammals. Within the cavity of the tubular structure, a bodily fluid flows to or from the body part. Examples of bodily fluid include blood, lymphatic fluid, or bile. Examples of vessels include arteries, arterioles, capillaries, venules, sinusoids, veins, lymphatics, and bile ducts. The intravascular route includes delivery through the blood vessels such as an artery or a vein. “Intracoronary” refers to an intravascular route for delivery to the heart wherein the blood vessels are the coronary arteries and veins.

Delivery of a nucleic acid means to transfer a nucleic acid from a container outside a mammal to near or within the outer cell membrane of a cell in the mammal. The term “transfection” is used herein, in general, as a substitute for the term “delivery,” or, more specifically, the transfer of a nucleic acid from directly outside a cell membrane to within the cell membrane. If the nucleic acid is a primary RNA transcript that is processed into messenger RNA, a ribosome translates the messenger RNA to produce a protein within the cytoplasm. If the nucleic acid is a DNA, it enters the nucleus where it is transcribed into a messenger RNA that is transported into the cytoplasm where it is translated into a protein. Therefore if a nucleic acid expresses its cognate protein, then it must have entered a cell. A protein may subsequently be degraded into peptides, which may be presented to the immune system.

A “therapeutic gene” refers herein to a nucleic acid that may have a therapeutic effect upon transfection into a cell. This effect can be mediated by the nucleic acid itself (e.g., anti-sense nucleic acid), following transcription (e.g., anti-sense RNA, ribozymes, interfering dsRNA), or following expression into a protein. “Protein” refers herein to a linear series of greater than 2 amino acid residues connected one to another as in a polypeptide. A “therapeutic” effect of the protein in attenuating or preventing the disease state can be accomplished by the protein either staying within the cell, remaining attached to the cell in the membrane, or being secreted and dissociated from the cell where it can enter the general circulation and blood. Secreted proteins that can be therapeutic include hormones, cytokines, growth factors, clotting factors, anti-protease proteins (e.g., alpha1-antitrypsin), angiogenic proteins (e.g., vascular endothelial growth factor, fibroblast growth factors), antiangiogenic proteins (e.g., endostatin, angiostatin), and other proteins that are present in the blood. Proteins on the membrane can have a therapeutic effect by providing a receptor for the cell to take up a protein or lipoprotein. Therapeutic proteins that stay within the cell (intracellular proteins) can be enzymes that clear a circulating toxic metabolite as in phenylketonuria. They can also cause a cancer cell to be less proliferative or cancerous (e.g., less metastatic), or interfere with the replication of a virus. Intracellular proteins can be part of the cytoskeleton (e.g., actin, dystrophin, myosins, sarcoglycans, dystroglycans) and thus have a therapeutic effect in cardiomyopathies and musculoskeletal diseases (e.g., Duchenne muscular dystrophy, limb-girdle disease). Other therapeutic proteins of particular interest to treating heart disease include polypeptides affecting cardiac contractility (e.g., calcium and sodium channels), inhibitors of restenosis (e.g., nitric oxide synthetase), angiogenic factors, and anti-angiogenic factors.

“Vectors” are nucleic acid molecules originating from a virus, a plasmid, or the cell of an organism into which another nucleic fragment of appropriate size can be integrated without loss of the vector's capacity for self-replication. Vectors introduce nucleic acids into host cells, where it can be reproduced. Examples are plasmids, cosmids, and yeast artificial chromosomes. Vectors are often recombinant molecules containing nucleic acid sequences from several sources. Vectors include viruses, for example adenovirus (an icosahedral (20-sided) virus that contains DNA; there are over 40 different adenovirus varieties, some of which cause respiratory disease), adeno-associated virus (AAV, a parvovirus that contains single stranded DNA), or retrovirus (any virus in the family Retroviridae that has RNA as its nucleic acid and uses the enzyme reverse transcriptase to copy its genome into the DNA and integrate into the host cell's chromosome).

“Viral gene transfer” is defined in this document as delivery of a viral particle into a cell by the normal means of entry of the particular virus. A viral particle is defined as the nucleic acid, the viral coat proteins and for some viruses the envelope that assemble inside an infected cell and then are able to infect another cell. Viral nucleic acid sequences alone are not defined as part of viral delivery. Enhancers, promoters, polyadenylation signals and genes such as thymidine kinase all originate from viruses and these are typically used in non-viral expression cassettes.

The process of delivering a nucleic acid to a cell has been commonly termed transfection or the process of “transfecting” and also it has been termed “transformation.” The term transfecting as used herein refers to the introduction of foreign DNA into cells. The nucleic acid could be used to produce a change in a cell that can be therapeutic. The delivery of nucleic acid for therapeutic and research purposes is commonly called “gene therapy.” The delivery of nucleic acid can lead to modification of the genetic material present in the target cell. The term “stable transfection” or “stably transfected” generally refers to the introduction and integration of foreign nucleic acid into the genome of the transfected cell. The term “stable transfectant” refers to a cell which has stably integrated foreign nucleic acid into the genomic DNA. Stable transfection can also be obtained by using episomal vectors that are replicated during the eukaryotic cell division (e.g., plasmid DNA vectors containing a papilloma virus origin of replication, artificial chromosomes). The term “transient transfection” or “transiently transfected” refers to the introduction of foreign nucleic acid into a cell where the foreign nucleic acid does not integrate into the genome of the transfected cell. The foreign nucleic acid persists in the nucleus of the transfected cell. The foreign nucleic acid is subject to the regulatory controls that govern the expression of endogenous genes in the chromosomes. The term “transient transfectant” refers to a cell which has taken up foreign nucleic acid but has not integrated this nucleic acid.

As used herein, the term “sample” is used in its broadest sense. Sample is meant to include a specimen or culture obtained from any source, including biological and environmental samples. Biological samples may be obtained from animals (including humans) and encompass fluids, solids, tissues, and gases. Biological samples include blood products, such as plasma, serum and the like. Environmental samples include environmental material such as surface matter, soil, water, crystals and industrial samples. These examples are not to be construed as limiting the sample types applicable to the present invention.

The following abbreviations are used herein: CMV, cytomegalovirus; CpG, dinucleotide of cytosine linked to guanine; PCR, polymerase chain reaction; pDNA, plasmid DNA; SEAP, secreted alkaline phosphatase.

II. Processes

The present invention relates to methods for expressing nucleic acids in cells in vivo. The methods comprise a means for obtaining long-term expression in cells. In preferred embodiments, the methods comprise delivery of linear nucleic acid molecules into cells. In preferred embodiments, the nucleic acid encodes a gene or a partial gene. The following description discusses preferred embodiments of the present invention. The present invention is not limited to these particular examples.

Several methods have been developed for the delivery of plasmid-encoded transgenes into mammalian cells in vivo, including vascular delivery under pressure [5]. Obtaining long-term expression of a transgene, however, has been problematic. Expression from supercoiled plasmids with the CMV promoter, for example, is very high one day following plasmid delivery (an average of 55 μg/ml human factor IX in mice injected with plasmid pMIR7), but by day 28 expression is undetectable (see Example 5). In contrast, we have been able to obtain expression of a transgene for at least 182 days when we used the same plasmid linearized. At 20 weeks post injection with the linearized pMIR7, an injected mouse expressed human factor IX at 482 ng/ml, 10% of the normal physiological level. This level of expression would be therapeutic in a hemophiliac. A study by Chen et al. using linearized plasmids corroborates our evidence that using linearized DNA can lead to long-term expression [11]. These researchers hypothesize that the mechanism involved concatemerization of the linear expression cassette. Their hypothesis is not in conflict with our data.

Plasmids contain bacterial sequences in the origin of replication and in the antibiotic-resistance gene. Bacterial DNA elicits an immune response in mice, due to both unmethylated CpG sequences and to some other (as yet unknown) aspect of the bacterial sequences. Mammalian genomic DNA has a greatly reduced frequency of the dinucleotide sequence CG compared to the statistical frequency of 1 in 16; this phenomenon is called CpG suppression. Most of the CpG dinucleotides in mammalian DNA are methylated on the 5 position of the cytosine. When a plasmid containing an expression cassette is linearized, the expression cassette that contains non-bacterial sequences can be isolated from the intervening bacterial sequences of the vector.

Expression cassettes that contain sequences to be transcribed in mammalian cells require a promoter of mammalian or viral origin, a sequence to be transcribed that can encode a messenger RNA (mRNA) for a gene or partial gene or can function as RNA, and signals for 3′ end formation of the RNA. For mRNA the 3′ signal is a polyadenylation signal. Additional sequences downstream of the coding region of a gene can affect transcription termination and translation of the mRNA. Small nuclear RNAs function as RNA and their genes contain 3′ end formation signals that differ from the polyadenylation signal.

In our experiments with plasmid pMIR7 encoding human factor IX, we linearized the plasmid with a restriction enzyme that left blunt ends. We are also using restriction enzymes that leave cohesive or non-cohesive overlapping (sticky) ends.

III. Methods of Use

A. A Process of Generating a Biologically Active Substance

The present invention provides a process for generating a substance that is biologically active in a cell. As used herein, the phrase “biologically active substance” means any substance having the ability to alter the function of a living cell, tissue or organism. A biologically active substance can be a drug or other therapeutic agent. A biologically active substance can also be a chemical that interacts with and alters the function of a cell. By way of example, a biologically active substance can be a protein or peptide fragment thereof such as a receptor agonist or antagonist. In addition, a biologically active substance can be a nucleic acid. The biologically active substance of the present invention is a linear nucleic acid. The DNA includes sequences that allow for expression in the cell. The linear DNA includes a promoter that is functional in the target cell and the promoter directs transcription of part of this DNA. In a preferred embodiment, the DNA includes sequences for translation of the transcript. The linear DNA can be obtained by restriction digestion of a plasmid using enzymes that generate blunt ends or sticky ends. One end can be blunt and the other end sticky. Sticky ends can be cohesive when generated by a single enzyme or they can differ (non-cohesive) when generated by two different enzymes. In another embodiment, the expression cassette can be prepared by polymerase chain reaction. The ends of the linear DNA prepared in such a manner may have blunt ends or may have an overhang of a single base at the 3′ end; such an overhang is usually a deoxyadenosine.

In one embodiment the linear DNA can have Tn5 transposase-recognition elements at one or both ends. The Tn5 elements can be the outer elements or inner elements, or mosaics of the outer and inner elements.

B. Process of Delivering a Biologically Active Nucleic Acid

A target cell (a cell to which the substance is to be delivered) is exposed to the biologically active nucleic acid of the present invention in the presence of a delivery system. The target cell is located in vivo (i.e., in a living organism). The biologically active substance and the delivery system are typically administered to the organism in such a way as to distribute those materials to the cell. The materials can be administered simultaneously or sequentially. The delivery system and biologically active substance can be infused into the cardiovascular system (e.g., intravenously, intraarterially), injected directly into tissue containing the target cell (e.g., intramuscularly), or administered via other parenteral routes well known to one skilled in the art.

### EXAMPLES

The following examples are provided in order to demonstrate and further illustrate certain preferred embodiments and aspects of the present invention and are not to be construed as limiting the scope thereof.

### Example 1

### Plasmid pMIR7

Plasmid pMIR7 encodes human factor IX driven by the cytomegalovirus promoter, and includes a chimeric intron, and the SV40 polyadenylation signal. It also contains a prokaryotic promoter and the SV40 promoter for the Kanamycin/Neomycin resistance gene, a bacterial origin of replication, and two Tn5 transposase binding elements that flank the other indicated elements. Supercoiled pMIR7 was grown in DH10B bacteria and isolated using a Qiagen Maxi Prep (EndoFree) kit.

### Example 2

### Preparation of Linear DNA with Blunt Ends

Plasmid pMIR7 (described in Example 1) was linearized exterior to the Tn5 elements with restriction enzyme PshA I to generate blunt ends. In a 500 μl reaction that was 1× for Takara Buffer K, 200 μg pMIR7 and 120 units of PshA I (Takara) were combined. This reaction was incubated at 37° C. for 20 hours. Another 5 μl of 12 units/μl PshA I were then added and the reaction was incubated for another 3 hours at 37° C. This reaction was then phenol:choroform:isoamyl alcohol extracted and ethanol precipitated using 2.3 M ammonium acetate and 2.3 volumes of ethanol. The DNA was resuspended in water at approximately 1 μg/μl.

### Example 3

### Intravascular (Tail Vein) Injections

Each mouse was injected with 2 ml saline solution containing 10 μg plasmid DNA (pDNA), either supercoiled plasmid or linearized plasmid as prepared in Example 2. Injections were carried out with high pressure, delivering the 2 ml solution into the tail vein in about 7 seconds [5].

### Example 4

### Factor IX Assay

Each mouse was bled from the retro-orbital sinus at various times after pDNA delivery. Cells were pelleted from the blood to obtain plasma. The plasma was evaluated for the presence of human factor IX by an ELISA test. Dilutions of pooled normal human plasma (George King Bio-Medical) were used to generate a standard curve.

### Example 5

### Human Factor TX Measured in Mouse Plasma Following Intravascular Injection of Plasmid DNA

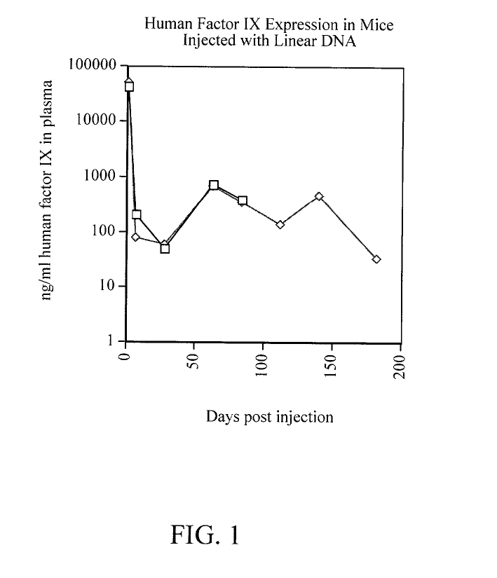

Mice were injected into the tail vein (according to Example 3) with either supercoiled or blunt-end linearized plasmid pMIR7 (prepared according to Example 2) encoding factor IX. Human factor IX (ng/ml) was measured in the plasma from mice at the indicated days post injection. Values were averaged from 4 mice for both DNA forms from day 1 and for blunt-linearized plasmid on days 7, 28 and 62. For supercoiled plasmid n=3 on days 7, 28 and 62. For two of the mice injected with the linearized pMIR7, human factor IX expression levels are shown for up to 182 days (FIG. 1). There was no detectable expression after day 7 from the mice injected with supercoiled pMIR7.

| | Human factor IX expression level | | | |
|---|---|---|---|---|
| | in mouse plasma (in ng/ml) at various | | | |
| | days after pDNA injection | | | |
| Injected DNA | Day 1 | Day 7 | Day 28 | Day 62 |
| Supercoiled plasmid | 55,620 | 9 | <1 | <1 |
| Blunt-linearized plasmid | 48,169 | 119 | 137 | 385 |

### Example 6

### Plasmid DNA with the EF1 Promoter

The CMV promoter in Example 1 can be replaced by the EF1 promoter to reduce the likelihood of promoter shut-down.

### Example 7

### Plasmid DNA Encoding Secreted Alkaline Phosphatase (SEAP)

As an alternative to the plasmid described in Examples 1 and 6, a plasmid encoding SEAP and driven by a eukaryotic promoter can be used to measure expression over time.

### Example 8

### Plasmid DNA with the Albumin Promoter

Plasmid pMIR142 contains a SEAP expression cassette as in Example 7. The mouse albumin promoter with a G to A point mutation at −53 drives the SEAP expression. The mouse alpha-fetoprotein enhancer II is positioned upstream of the promoter. This plasmid also contains the bacterial origin of replication and the kanamycin-resistance gene.

### Example 9

### Linear DNA with Compatible Cohesive Ends

Plasmid DNA such as Example 1, Example 6, Example 7 or Example 8 can be linearized with restriction enzymes that generate staggered ends in order to test whether linear DNA with cohesive termini can be used as well as linear DNA with blunt ends to bring about long-term expression. Plasmid DNA can be cut with a restriction enzyme such as Bgl II to generate compatible sticky ends.

### Example 10

### Linear DNA with Incompatible Sticky Ends

Plasmid DNA such as Examples 1, 6, 7 or 8 can be cut with two different restriction enzymes to generate incompatible sticky ends.

### Example 11

### Isolated Expression Cassette

Plasmid pMIR142 as in Example 8 is linearized with Pac I and Sse8387 I as in Example 10 to separate the expression cassette from the intervening bacterial sequences. The expression cassette is isolated by gel purification from low melting point agarose and then recovered with the GELase protocol (Epicentre, Madison, Wis.).

### Example 12

### SEAP Assay

Plasmid DNA encoding SEAP (such as in Example 7, 8 or 11) can be prepared by linearizing to generate blunt ends (according to Example 2) or sticky ends (as in Examples 9, 10 or 11). Mice are injected in the tail vein (according to Example 3) with the DNA samples. Each mouse is bled from the retro-orbital sinus at various times after DNA delivery. Cells and clotting factors are pelleted from the blood to obtain serum. The serum is evaluated for the presence of SEAP by a chemiluminescence assay using the Tropix Phospha-Light kit.

### Example 13

### Use of Mice with Lower Immune Response

To reduce the immune response of the mice to the expressed product of the transgene (human factor IX in Examples 1 and SEAP in Example 7), C57B1/6 or SCID Beige mice are used.

The foregoing is considered as illustrative only of the principles of the invention. Furthermore, since numerous modifications and changes will readily occur to those skilled in the art, it is not desired to limit the invention to the exact construction and operation shown and described. Therefore, all suitable modifications and equivalents fall within the scope of the invention.

We claim:

1. A process for in vivo transgene expression, comprising:
a. delivering a non-viral, linear nucleic acid to a cell, in vivo; and,
b. expressing the nucleic acid for extended periods of time.

2. The process of claim 1, wherein the nucleic acid contains blunt ends.

3. The process of claim 1, wherein the nucleic acid contains sticky ends.

4. The process of claim 1, wherein the nucleic acid contains a blunt end and a sticky end.

5. The process of claim 1, wherein the linear nucleic acid is generated by restriction enzyme digestion.

6. The process of claim 1, wherein the linear nucleic acid is generated by polymerase chain reaction.

7. The process of claim 1, wherein the nucleic acid contains an expression cassette isolated from a plasmid backbone.

8. The process of claim 1, wherein the nucleic acid contains an expression cassette which is flanked by inside ends derived from Tn5 transposase.

9. The process of claim 8, wherein the nucleic acid ends are blunt.

10. The process of claim 1, wherein the nucleic acid contains an expression cassette which is flanked by outside ends derived from Tn5 transposase.

11. The process of claim 10, wherein the nucleic acid ends are blunt.

12. The process of claim 1, wherein the nucleic acid contains an expression cassette which is flanked by chimeric ends derived from Tn5 transposase.

13. The process of claim 12, wherein the nucleic acid ends are blunt.

14. The process of claim 1, wherein the nucleic acid is delivered to cells intravascularly.

15. The process of claim 1, wherein the nucleic acid are delivered intravascularly using pressure.

16. The process of claim 1, wherein the nucleic acid is delivered by direct intramuscular injection.

17. The process of claim 1, wherein the nucleic acid is delivered by direct interstitial injection.

18. A process for transgene expression, comprising:
a. generating expression cassettes from a non-viral, linear vector,
b. expressing the nucleic acid for extended periods of time.

19. The process of claim 18, wherein the linear nucleic acid is prepared by restriction enzyme digestion.

20. The process of claim 18, wherein the linear nucleic acid is prepared by polymerase chain reaction.
